(19) United States
(12) Patent Application Publication
(10) Pub. No.: US 2018/0208882 A1
(43) Pub. Date: Jul. 26, 2018

(54) BEER-TASTE BEVERAGE, METHOD FOR PRODUCING BEER-TASTE BEVERAGE, AND METHOD FOR IMPROVING AROMA OF BEER-TASTE BEVERAGE

(71) Applicant: SAPPORO HOLDINGS LIMITED, Shibuya-ku (JP)

(72) Inventors: Takeo ISHIHARA, Tokyo (JP); Tetsu HAMAGUCHI, Tokyo (JP)

(73) Assignee: SAPPORO HOLDINGS LIMITED, Shibuya-ku (JP)

(21) Appl. No.: 15/744,533

(22) PCT Filed: Sep. 14, 2016
(86) PCT No.: PCT/JP2016/077152

§ 371 Date: Jan. 12, 2018

(30) Foreign Application Priority Data
Sep. 16, 2015 (JP) 2015-183417

(51) Int. Cl. C12C 5/02; C12C 5/04; C12G 3/06; C12G 3/08; C07C 69/14

(52) U.S. Cl. CPC C12C 5/026; C12C 5/04; C07C 69/14; C12G 3/08; C12G 3/06

(57) ABSTRACT

Provided is a beer-taste beverage in which oxidized soy sauce odor is changed to good aroma, a method for producing the beer-taste beverage, and a method for improving aroma of the beer-taste beverage. The beer-taste beverage according to the present invention has an a* value in an L*a*b* color system of 7.0 to 21.0, and an acetate ester content of 0.7 to 2.4 mg/L. The method for producing the beer-taste beverage according to the present invention includes producing the beverage so that the a* value in the L*a*b* color system is 7.0 to 21.0 and the acetate ester content is 0.7 to 2.4 mg/L. In the method for improving the aroma of the beer-taste beverage according to the present invention, the beer-taste beverage is prepared so that the a* value in the L*a*b* color system is 7.0 to 21.0 and the acetate ester content is 0.7 to 2.4 mg/L.

### TECHNICAL FIELD

The present invention relates to a beer-taste beverage, a method for producing the beer-taste beverage, and a method for improving aroma of the beer-taste beverage.

### BACKGROUND ART

Although the beer-taste beverage generally has golden or amber color, there are beer-taste beverages having various colors such as black, red and white. Therefore, a consumer can select the beer-taste beverage considering not only flavor but also color.

Various researches and developments have been made on the color of the beer-taste beverage, the color being a point in selecting the beer-taste beverage. For example. Patent Document 1 discloses a fermented malt beverage produced by a production method in which a pre-fermentation liquid containing Maillard reaction products and colored malt-containing malt is fermented by beer yeast (in the production method, liquid color of the fermented malt beverage is adjusted using an amount of 3-deoxyglucosone in the beverage as an index), wherein the beverage has a chromaticity (EBC) of 8 to 12 and a content of 3-deoxyglucosone of 25 ppm to 50 ppm.

### CITATION LIST

### Patent Literature

[Patent Document 1]

Japanese Patent No. 5474756

### SUMMARY OF INVENTION

### Technical Problem

A technique disclosed in Patent Document 1 is intended to bring the color and flavor of the fermented malt beverage close to color and flavor of lager type beer by controlling the chromaticity and the content of 3-deoxyglucosone within predetermined ranges (paragraph 0036 of Patent Document 1).

The present inventors have studied production of the beer-taste beverage having red color rather than the color of the lager type beer as disclosed in Patent Document 1, and conducted tests on a large number of samples under various conditions such as raw materials. During the tests, the present inventors found a problem that the higher a degree of red color of the beer-taste beverage is, the stronger odor (brown odor) like oxidized soy sauce odor is felt, and this odor adversely affects an impression of the beer-taste beverage.

Therefore, an object of the present invention is to provide a beer-taste beverage in which an oxidized soy sauce odor is changed to good aroma, a method for producing the beer-taste beverage, and a method for improving aroma of the beer-taste beverage.

### Solution to Problem

The above problems can be solved by the following means.

(1) A beer-taste beverage, wherein an a* value in an L*a*b* color system is 7.0 or more and 21.0 or less, and content of acetate ester is 0.7 mg/L or more and 2.4 mg/L or less.
(2) The beer-taste beverage according to the above (1), wherein the a* value is 11.0 or more.
(3) The beer-taste beverage according to the above (1) or (2), wherein the a* value is 16.0 or less.
(4) The beer-taste beverage according to any one of the above (1) to (3), wherein the content of the acetate ester is 1.0 mg/L or more and 2.0 mg/L or less.
(5) The beer-taste beverage according to any one of the above (1) to (4), wherein the acetate ester is isoamyl acetate.
(6) The beer-taste beverage according to any one of the above (1) to (5), wherein an L* value is 50.0 or more and 90.0 or less, and a b* value is 50.0 or more and 90.0 or less.
(7) The beer-taste beverage according to any one of the above (1) to (6), wherein no colorant is contained therein.
(8) The beer-taste beverage according to any one of the above (1) to (7), wherein a malt ratio is 50 mass % or more.
(9) A beer-taste beverage, wherein an a* value in an L*a*b* color system is 11.0 or more and 16.0 or less, content of acetate ester is 1.5 mg/L or more and 2.0 mg/L or less, the acetate ester is isoamyl acetate, an L* value is 50.0 or more and 90.0 or less, a b* value is 50.0 or more and 90.0 or less, no colorant is contained therein, and a malt ratio is 50 mass % or more.
(10) A method for producing a beer-taste beverage, wherein the beer-taste beverage is produced so that an a* value in an L*a*b* color system is 7.0 or more and 21.0 or less, and content of acetate ester is 0.7 mg/L or more and 2.4 mg/L or less.
(11) A method for improving aroma of a beer-taste beverage, wherein the beer-taste beverage is prepared so that an a* value in an L*a*b* color system is 7.0 or more and 21.0 or less, and content of acetate ester is 0.7 mg/L or more and 2.4 mg/L or less.

### Advantageous Effects of Invention

The beer-taste beverage according to the present invention is a beer-taste beverage in which the oxidized soy sauce odor is changed to fragrance (good aroma) like dark cherry.

By a method for producing the beer-taste beverage according to the present invention, it is possible to produce the beer-taste beverage in which the oxidized soy sauce odor is changed to fragrance (good aroma) like dark cherry.

By a method for improving aroma of the beer-taste beverage according to the present invention, it is possible to change the oxidized soy sauce odor of the beer-taste beverage to fragrance (good aroma) like dark cherry.

### BRIEF DESCRIPTION OF DRAWINGS

FIG. 1 is a flowchart illustrating a method for producing a beer-taste beverage according to an embodiment of the present invention.

### DESCRIPTION OF EMBODIMENTS

Hereinafter, embodiments of a beer-taste beverage according to the present invention, a method for producing the beer-taste beverage, and a method for improving aroma of the beer-taste beverage will be described.

### [Beer-Taste Beverage]

The beer-taste beverage according to the present invention has an a* value in an L*a*b* color system in a predetermined range and a content of acetate ester in a predetermined range. Here, the beer-taste beverage means a beverage having a beer-like taste, that is, a beverage giving drinkers a feeling as if drinking beer. The beer-taste beverage includes a beverage (also referred to as a beer-taste non-alcoholic beverage, a non-alcoholic beer-taste beverage or the like) having an alcohol content of less than 1% (also referred to as vol/vol %, v/v % or the like) and a beverage (referred to as a beer-taste alcoholic beverage or the like) having an alcohol content of 1% or more. The beer-taste beverage may be a fermented beer-taste beverage not using malt, wheat or the like as raw materials (for example, a beverage classified as “other brewed alcohol (foaming) (1)” under the liquor tax law), or a fermented malt beverage such as a beer, a foaming liquor or a liqueur using malt as the raw materials (for example, a beverage classified as “liquor (foaming) (1)” under the liquor tax law). A malt ratio (malt use ratio) of the fermented malt beverage is preferably 50 mass % or more, and more preferably 67 mass % or more.

### (L*a*b* Color System)

The beer-taste beverage according to the present embodiment has the a* value in the L*a*b* color system of 7.0 to 21.0. Here, the L*a*b* color system is an index used to indicate color of an object. The L*a*b* color system is standardized by the International Commission on Illumination (CIE) in 1976, and is specified in JIS Z8781-4:2013. In the L*a*b* color system, lightness is indicated by L* value, and chromaticity (hue and chroma) is indicated by a* value and b* value. The larger the L* value is, the brighter it is. The a* and b* indicate directions of color, and a* indicates a red direction, −a* indicates a green direction, b* indicates a yellow direction, and −b* indicates a blue direction.

When the a* value in the L*a*b* color system is 7.0 to 21.0, the beer-taste beverage has a red color and a problem to be solved appears that oxidized soy sauce odor is felt. In the present invention, the oxidized soy sauce odor and acetate ester (aroma derived from acetate ester) to be described below act synergistically and they can act more synergistically when the a* value is within the predetermined range. In order to increase a degree of red color and to allow the oxidized soy sauce odor and the acetate ester to act more synergistically, the a* value is preferably 11.0 or more. On the other hand, an upper limit of the a* value in the L*a*b* color system is preferably 20.0 or less and more preferably 16.0 or less in consideration of a balance of aroma and a synergistic interaction between the oxidized soy sauce odor and the acetate ester.

The L* value in the L*a*b* color system is not particularly limited, however, in order to make the red color a more suitable bright color for the beer-taste beverage, a lower limit of the L* value is preferably 50.0 or more and more preferably 54.0 or more, and an upper limit of the L* value is preferably 90.0 or less and more preferably 81.0 or less. The b* value in the L*a*b* color system is not particularly limited, however, in order to make the red color a more suitable red color for the beer-taste beverage, a lower limit of the b* value is preferably 50.0 or more and more preferably 53.0 or more, and an upper limit of the b* value is preferably 90.0 or less and more preferably 85.0 or less.

Each value in the L*a*b* color system can be controlled by selecting the raw materials such as malt or by changing production conditions. The a* value can be increased, for example, by increasing a use ratio of malt (color malt) having a deep color due to high roasting temperature or the like, or by increasing boiling time of pre-fermentation liquid (wort, preparation liquid). The a* value can also be increased by increasing a ratio of post-fermentation liquid having a large a* value when mixing the post-fermentation liquid having a large a* value and the post-fermentation liquid having a small a* value. Each value in the L*a*b* color system of the beer-taste beverage can be obtained, for example, by transparent object color measurement using a spectrophotometer (device name: CM-3600d manufactured by Konica Minolta, Inc.).

### (Acetate Ester)

The beer-taste beverage according to the present embodiment contains acetate ester of 0.7 to 2.4 mg/L. Here, the acetate ester is an ester produced from acetic acid and alcohol and is represented by general formula CH₃COOR. The acetate ester is preferably at least one of ethyl acetate (R in the general formula is C₂H₅) and isoamyl acetate (R in the general formula is C₅H₁₁) and more preferably isoamyl acetate.

When the content of acetate ester is 0.7 mg/L or more, the oxidized soy sauce odor and the acetate ester (aroma derived from the acetate ester) act synergistically, so that the oxidized soy sauce odor can be changed to fragrance like dark cherry. In order to obtain more preferable aroma, the content of acetate ester is preferably 1.0 mg/L or more, more preferably 1.3 mg/L or more, and even more preferably 1.5 mg/L or more. On the other hand, when the content of acetate ester is 2.4 mg/L or less, the oxidized soy sauce odor and the acetate ester (aroma derived from the acetate ester) can act appropriately. In order to obtain more preferable aroma, the content of acetate ester is preferably 2.0 mg/L or less.

The content of acetate ester can be controlled (increased) by increasing fermentation time in a production step (fermentation step S2) to be described below, by using yeast having a high ability to produce acetate ester, or by increasing an amino acid content or a dissolved oxygen content in the pre-fermentation liquid (won, preparation liquid). Further, the content of acetate ester can also be increased by separately adding the acetate ester itself. Furthermore, the content of acetate ester can also be increased by increasing a ratio of the post-fermentation liquid containing a large amount of acetate ester when mixing the post-fermentation liquid containing a large amount of acetate ester and the post-fermentation liquid containing a small amount of acetate ester. The content of acetate ester in the beer-taste beverage according to the present embodiment can be measured using a gas chromatograph (device name: Agilent 6890 gas chromatograph) equipped with an FID detector, according to a method in “8.22 Low boiling aroma component” of BCOJ beer analysis method (published by the Brewing Society of Japan, edited by the Brewers Association International Technology Committee (Analysis Committee), 1 Apr. 1996).

### (Colorant)

The beer-taste beverage according to the present embodiment preferably contains no colorant. Here, the colorant is a dye to be added only for coloring the beer-taste beverage, and a dye derived from the raw materials used in a production step (pre-fermentation step S1) to be described below is not a colorant because it is not separately added for coloring the beer-taste beverage. In particular, the colorant is caramel pigment, anthocyanin, gardenia pigment, fruit juice dyes, vegetable dyes, synthetic dyes or the like. Since the beer-taste beverage according to the present embodiment contains no colorant, it has an original natural color of wheat or the like.

### (Foaming Property)

The beer-taste beverage according to the present embodiment may be non-foaming, but is preferably foaming. Here, foaming property in the present embodiment means that a gas pressure at 20° C. is 0.049 MPa (0.5 kg/cm²) or more, and non-foaming property means that the gas pressure at 20° C. is less than 0.049114 Pa (0.5 kg/cm²).

### (Other)

Bitterness unit of the beer-taste beverage according to the present embodiment is not particularly limited. The bitterness unit may be derived from the raw materials such as hop or may be added as a hop product.

As the hop product, for example, hop pellets obtained by a pre-grinding process, hop pellets obtained by screening lupulin grains in advance during the pre-grinding process and containing a lot of lupulin, and hop extract obtained by extracting, for example, essential oil and bitter substance of the lupulin can be used. As a method of adding the hop, there is, for example, kettle hopping, late hopping or dry hopping, however, the method is not limited thereto. Here, the kettle hopping refers to adding the hop during rise in temperature of pre-fermentation liquid (wort) or early in the boiling of the wort, and the late hopping refers to adding the hop just before the end of the boiling of the wort. Further, the dry hopping refers to adding the hop after the beginning of the fermentation step.

As the hop product, for example, low bop, hexa-hop, tetra-hop or isomerized hop extract can be used. The hop product can be added in any one or more steps of pre-fermentation step S1 to post-fermentation step S3.

In the beer-taste beverage according to the present embodiment, purine content and content of extracted components derived from wheat or the like are not particularly limited. Here, the extracted components derived from wheat or the like only have to be derived from wheat or the like, and form of the raw materials (derived from wheat or the like) is not limited. As the form of the raw materials derived from wheat or the like, there are, for example, wheat or the like, malt and extract from them, and one or more of the forms can be used. The wheat or the like, the malt and the extract from them are obtained by appropriately processing barley, wheat, rye, oat and the like. The wheat or the like have a great influence on fragrance and taste of the beer-taste beverage, and are nitrogen source and carbon source assimilable by yeast in alcohol fermentation.

The wheat or the like used as the raw materials derived from wheat or the like, refer to barley, wheat, rye, oat or the like, which is not germinated. The wheat or the like may be threshed or their grains may be used as they are or, for example, by being crushed in appropriate sizes. The malt used as the raw materials derived from wheat or the like refers to barley, wheat, rye, oat and the like, which are germinated under predetermined conditions. The malt may be used by being germinated or, for example, by being crushed in appropriate sizes. The extract derived from the malt or the wheat or the like refers to extract obtained, for example, by extracting predetermined components from the malt or the wheat or the like by using, for example, water and/or organic solvent, and by concentrating the components. The above-described raw materials derived from wheat or the like may also be used after roasting under various conditions so that the a* value in the L*a*b* color system can be within the predetermined range.

The beer-taste beverage according to the present embodiment can be prepared, if necessary, by further adding distilled alcohol to alcohol obtained by fermenting the raw materials derived from wheat or the like. The distilled alcohol includes, for example, material alcohol and various spirits such as shochu (distilled spirits), brandy, vodka and whiskey. One distilled alcohol may be used alone, or two or more types of alcohols may be used in combination. In the present specification, “spirits” refers to spirits which is distilled liquor, and is different from spirits under the liquor tax law in some cases.

The beer-taste beverage according to the present embodiment can also be added with, for example, sweetener, high intensity sweetener, antioxidant, flavoring agent, acidulant, salts or dietary fiber (hereinafter, appropriately referred to as an “additive”), which is usually blended into the beverage, to the extent that desired effects of the present invention are not impaired. As the sweetener, it is possible to use, for example, high fructose corn syrup, glucose, galactose, mannose, fructose, lactose, sucrose, maltose, glycogen or starch. As the high intensity sweetener, it is possible to use, for example, neotame, acesulfame potassium, sucralose, saccharin, sodium saccharin, disodium glycyrrhizinate, cyclamate, dulcin, stevia, glycyrrhizin, thaumatin, monellin, aspartame or alitame. As the antioxidant, it is possible to use, for example, vitamin C, vitamin E or polyphenols. As the acidulant, it is possible to use, for example, adipic acid, citric acid, trisodium citrate, glucono-δ-lactone, gluconic acid, potassium gluconate, sodium gluconate, succinic acid, monosodium succinate, disodium succinate, sodium acetate, DL-tartaric acid, L-tartaric acid, DL-sodium tartrate, L-sodium tartrate, carbon dioxide, lactic acid, sodium lactate, glacial acetic acid, fumaric acid, monosodium fumarate, DL-malic acid, DL-sodium malate or phosphoric acid. As the salts, it is possible to use, for example, sodium chloride, acidic potassium phosphate, acidic calcium phosphate, ammonium phosphate, magnesium sulfate, calcium sulfate, potassium metabisulfite, calcium chloride, magnesium chloride, potassium nitrate or ammonium sulfate. As the dietary fiber, it is possible to use, for example, indigestible dextrin, pectin, polydextrose or guar gum hydrolysate. As the additive described above, those commercially available can be generally used.

### (Packaged Beer-Taste Beverage)

The beer-taste beverage according to the present embodiment can be provided in various containers. By filling the beer-taste beverage in various containers, it is possible to suitably prevent quality deterioration due to long term storage. The container only has to be a hermetically sealable one, and it is possible to use so-called can containers or barrel containers made of metal (aluminum, steel or the like). Further, it is also possible to use glass containers, PET bottle containers, paper containers, pouch containers or the like. Volume of the container is not particularly limited, and it is possible to use any one currently available. The container made of metal is preferably used in view of completely blocking gas, water and light and maintaining stable quality for long periods at ambient temperature.

As described above, with the beer-taste beverage according to the present embodiment, since the a* value in the L*a*b* color system is within the predetermined range and the content of acetate ester is within the predetermined range, the oxidized soy sauce odor is changed to the fragrance (good aroma) like dark cherry.

### [Method for Producing Beer-Taste Beverage]

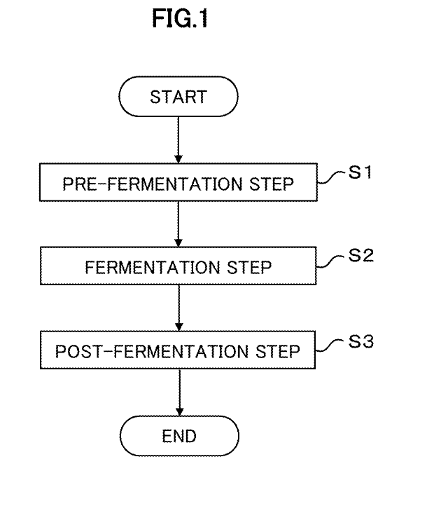

Next, the method for producing the beer-taste beverage according to the present embodiment will be described. As shown in FIG. 1, the method for producing the beer-taste beverage according to the present embodiment includes pre-fermentation step S1, fermentation step S2 and post-fermentation step S3.

### (Pre-Fermentation Step S1)

The pre-fermentation step S1 is a step of preparing the pre-fermentation liquid containing the raw materials derived from wheat or the like. The pre-fermentation liquid prepared in the pre-fermentation step S1 is not particularly limited as long as it is a solution containing the raw materials derived from wheat or the like, which are the nitrogen source and carbon source assimilable by yeast. The nitrogen source and carbon source are not particularly limited as long as they are assimilable by yeast. The nitrogen source assimilable by yeast is, for example, at least one of amino acids and peptides contained in the raw materials derived from wheat or the like. The carbon source assimilable by yeast is, for example, sugars contained in the raw materials derived from wheat or the like. Since the raw materials derived from wheat or the like have been already described in detail, the description is omitted.

When the pre-fermentation liquid contains the raw materials derived from wheat or the like, it is preferable to perform a step (so-called saccharification step) of decomposing protein and/or polysaccharides contained in the raw materials by enzymes in the pre-fermentation liquid. As the enzymes, there are, for example, protease and/or amylase. The enzymes contained in the raw materials derived from wheat or the like may be used, and the enzymes previously refined may be externally added instead of or in addition to the enzymes contained in the raw materials.

The pre-fermentation liquid is preferably filtered and is more preferably boiled before fermentation by yeast in the succeeding fermentation step S2. By filtering the pre-fermentation liquid, contaminants can be eliminated, so that the beer-taste beverage having higher quality can be provided. Further, the pre-fermentation liquid can be sterilized to be a sterile state by boiling, so that alcohol fermentation in the fermentation step S2 can be appropriately performed.

### (Fermentation Step S2)

The fermentation step S2 is a step of performing alcohol fermentation by adding yeast to the pre-fermentation liquid. In the present embodiment, for example, at first the fermentation liquid is prepared by adding yeast to the pre-fermentation liquid in a sterile state, which is previously prepared within a predetermined range of temperature (for example, a range of 0 to 40° C.).

The number of yeasts in the fermentation liquid at a start of fermentation can be appropriately adjusted, for example, within a range of 1×10² to 3×10⁹ cells/mL, and preferably within a range of 1×10⁶ to 3×10⁹ cells/mL.

Then, fermentation is performed by maintaining the fermentation liquid at a predetermined temperature for a predetermined time. Temperature of fermentation can be appropriately adjusted, for example, within a range of 0 to 40° C. and preferably within a range of 6 to 15° C.

In the fermentation step S2, aging may be further performed. Aging is performed by further maintaining the fermentation liquid after fermentation described above at a predetermined temperature for a predetermined time. By this aging, it is possible to remove turbidity by precipitating insoluble matter in the fermentation liquid, and to improve flavor.

Thus, in the fermentation step S2, it is possible to obtain post-fermentation liquid containing flavor components and ethanol produced by yeast. Concentration of ethanol (alcohol content) contained in the post-fermentation liquid can be, for example, 1 to 20%. When reducing the alcohol content to less than 1%, it can be achieved by appropriately adjusting fermentation condition, for example, by shortening fermentation time in the fermentation step S2 or by lowering fermentation temperature. The alcohol content can also be reduced to less than 1% by appropriately diluting the post-fermentation liquid having an alcohol content of 1 to 20%.

### (Post-Fermentation Step S3)

The post-fermentation step S3 is a step of finally obtaining the beer-taste beverage by subjecting the post-fermentation liquid to a predetermined process. As the post-fermentation step S3, for example, there is filtration (so-called primary filtration) of the post-fermentation liquid obtained by the fermentation step S2. By the primary filtration, it is possible to remove insoluble solid content and yeast from the post-fermentation liquid. In the post-fermentation step S3, microfiltration (so-called secondary filtration) of the post-fermentation liquid may also be further performed. By the secondary filtration, it is possible to remove bacteria and remaining yeast from the post-fermentation liquid. It is also possible to perform sterilization by heating the post-fermentation liquid instead of microfiltration. Primary filtration, secondary filtration and heating in the post-fermentation step S3 can be performed in general facilities used in production of the beer-taste beverage.

The beer-taste beverage can be produced so that the a* value and the content of acetate ester of the beer-taste beverage obtained by the post-fermentation step S3 are within the predetermined ranges described above. When the a* value and the content of acetate ester of the beer-taste beverage obtained by the post-fermentation step S3 are not within the predetermined ranges described above, the following processes may be performed in the post-fermentation step S3. The beer-taste beverage may be prepared so that the a* value and the content of acetate ester of the beer-taste beverage finally obtained are within the predetermined ranges described above, for example, by mixing the post-fermentation liquid having a large a* value and the post-fermentation liquid having a small a* value, by mixing the post-fermentation liquid containing a large amount of acetate ester and the post-fermentation liquid containing a small amount of acetate ester, or by adding the acetate ester itself to the post-fermentation liquid.

Further, when it is desired to increase the alcohol content of the post-fermentation liquid, the alcohol, that is, the spirits or the like is preferably added in the post-fermentation step S3. When the produced beer-taste beverage is non-foaming, or the foaming property is not sufficient, and it is desired to increase the foaming property of the beer-taste beverage sufficiently, the gas pressure may be increased to a desired pressure by carbonation or by adding water containing carbon dioxide in the post-fermentation step S3. The post-fermentation step S3 includes a step of filling the container with the beer-taste beverage.

The beer-taste beverage according to the present embodiment can be produced without going through the fermentation step S2. For example, the beer-taste beverage can be also produced by being added and mixed with the wort, the hop extract, the acetate ester, the additive or the like. In this case, each value in the L*a*b* color system can be controlled by selecting the raw materials such as malt and by changing production conditions. The a* value can also be controlled by changing a mixing ratio of the wort having a large a* value and the wort having a small a* value. The a* value can further be controlled by adding a colorant, however, as described above, the colorant is preferably not added. The content of acetate ester can be controlled by an amount of acetate ester itself to be added, and also by changing a mixing ratio of the wort having a large content of acetate ester and the wort having a small content of acetate ester.

As described above, by the method for producing the beer-taste beverage according to the present embodiment, the beer-taste beverage is produced so that the a* value in the L*a*b* color system is within the predetermined range and the content of acetate ester is within the predetermined range, and thus it is possible to produce the beer-taste beverage in which the oxidized soy sauce odor is changed to the fragrance (good aroma) like dark cherry.

### [Method for Improving Aroma of Beer-Taste Beverage]

Next, the method for improving aroma of the beer-taste beverage according to the present embodiment will be described. The method for improving aroma of the beer-taste beverage according to the present embodiment is characterized in that the beer-taste beverage is prepared so that the a* value in the L*a*b* color system is within the predetermined range and the content of acetate ester is within the predetermined range.

In the method for improving aroma of the beer-taste beverage according to the present embodiment, the beer-taste beverage which is a final product is prepared so that the a* value in the L*a*b* color system is 7.0 to 21.0. A lower limit of the a* value is preferably 11.0 or more. On the other hand, the upper limit of the a* value in the L*a*b* color system is preferably 20.0 or less and more preferably 16.0 or less.

In the method for improving aroma of the beer-taste beverage according to the present embodiment, the beer-taste beverage which is the final product is prepared so that the content of acetate ester is 0.7 to 2.4 mg/L. A lower limit of the content of acetate ester is preferably 1.0 mg/L or more, more preferably 1.3 mg/L or more, and even more preferably 1.5 mg/L or more. On the other hand, an upper limit of the content of acetate ester is preferably 2.0 mg/L or less.

As described above, by the method for improving aroma of the beer-taste beverage according to the present embodiment, the beer-taste beverage is prepared so that the a* value in the L*a*b* color system is within the predetermined range and the content of acetate ester is within the predetermined range, and thus it is possible to change the oxidized soy sauce odor to the fragrance (good aroma) like dark cherry.

With respect to the beer-taste beverage, the method for producing the beer-taste beverage, and the method for improving aroma of the beer-taste beverage according to the present embodiment, the properties and conditions not explicitly described may be those conventionally known, and it goes without saying that they are not limited as long as the effects by the properties and conditions are obtained.

### EXAMPLES

Next, by showing examples satisfying requirements of the present invention and comparative examples not satisfying the requirements of the present invention, the beer-taste beverage, the method for preparing the beer-taste beverage, and the method for improving aroma of the beer-taste beverage according to the present invention will be described.

### REFERENCE EXAMPLES

First, in reference examples, influence of the a* value of the beer-taste beverage on “the oxidized soy sauce odor (brown odor)” is checked.

### (Preparation of Samples)

By the method for preparing the beer-taste beverage according to the present embodiment, samples (beer-taste beverages) are prepared so that they have the a* values and the contents of isoamyl acetate shown in Table 1. The alcohol contents of the samples are 5 to 6%, and the gas pressures of the samples are substantially equal to each other. No colorant is added separately to the samples, and further no flavoring agent other than isoamyl acetate is added thereto.

### (Oxidized Soy Sauce Odor: Evaluation Method)

Three trained expert panel members independently scored the oxidized soy sauce odor of the samples prepared by the method described above on a scale of 1 to 5 according to the following evaluation criteria, and calculated their average values. With respect to “the oxidized soy sauce odor”, the fragrance (vaporized fragrance) felt when bringing one's nose close to the sample was checked and evaluated.

### (Oxidized Soy Sauce Odor: Evaluation Criteria)

5 points: oxidized soy sauce odor is very strong

4 points: oxidized soy sauce odor is strong

3 points: oxidized soy sauce odor is felt

2 points: oxidized soy sauce odor is slightly felt

1 point: oxidized soy sauce odor is not felt at all

Table 1 shows specifications of the samples and evaluation results of the samples.

TABLE 1

| | Sample No. | | | | |
|---|---|---|---|---|---|
| | 1-1 | 1-2 | 1-3 | 1-4 | 1-5 |
| Isoamyl acetate (mg/L) | 0.8 | 0.4 | 0.4 | 0.4 | 0.4 |
| a* value | 0 | 5 | 12 | 19 | 26 |
| Oxidized soy sauce | 1.0 | 3.0 | 4.0 | 5.0 | 5.0 |
| odor (brown odor) | | | | | |

### Study of Test Results: Reference Examples

As is apparent from the results of Samples 1-1 to 1-5, it is confirmed that the oxidized soy sauce odor becomes stronger as the a* value increases. From this results, it is found that there is a problem that the oxidized soy sauce odor becomes stronger as the a* value increases. Since Samples 1-1 to 1-5 are produced without adding any colorant, it is presumed that the oxidized soy sauce odor is due to the a* value derived from the color malt (raw material).

### EXAMPLES

Next, in the examples, effect of the a* value and the content of isoamyl acetate of the beer-taste beverage on evaluation is checked.

### (Preparation of Samples)

By the method for producing the beer-taste beverage according to the present embodiment, samples were prepared so that they have the a* values and the contents of isoamyl acetate shown in Tables 2, 3. The alcohol contents of the samples are 5 to 6%, and the gas pressures of the samples are substantially equal to each other. No colorant is added separately to the samples, and further no flavoring agent other than isoamyl acetate is added thereto. The malt ratios of the samples are 67% or more.

### (Fragrance Like Dark Cherry: Evaluation Method)

Three trained expert panel members independently scored the fragrance like dark cherry of the samples prepared by the method described above on a scale of 1 to 5 according to the following evaluation criteria, and calculated their average values. With respect to “the fragrance like dark cherry”, the fragrance (vaporized fragrance) felt when bringing one's nose close to the sample was checked and evaluated.

### (Fragrance Like Dark Cherry: Evaluation Criteria)

5 points: very desirable fragrance like dark cherry is felt

4 points: desirable fragrance like dark cherry is felt

3 points: fragrance like dark cherry is felt

2 points: fragrance like dark cherry is felt but weak

1 point: fragrance like dark cherry is not felt

### (Appearance of Red Beer: Evaluation Method)

Three trained expert panel members independently scored appearance of red beer of the samples prepared by the method described above on a scale of 1 to 5 according to the following evaluation criteria, and calculated their average values. With respect to “the appearance of red beer (red beer-taste beverage)”, the sample was poured into a transparent cup and the appearance (color) was checked and evaluated.

### (Appearance of Red Beer: Evaluation Criteria)

5 points: red color is very strong and looks very much like red beer

4 points: red color is strong and looks quite like red beer

3 points: color is red and looks like red beer

2 points: red color is weak but is recognized as red beer

1 point: red color is not recognized and not recognized as red beer

Tables 2, 3 show specifications of the samples and evaluation results of the samples.

TABLE 2

| | Sample No. | | | | | |
|---|---|---|---|---|---|---|
| | 2-1 | 2-2 | 2-3 | 2-4 | 2-5 | 2-6 |
| Isoamyl acetate (mg/L) | 0.4 | 0.7 | 1 | 1.5 | 2 | 2.5 |
| a* value | 11 | 11 | 11 | 11 | 11 | 11 |
| Fragrance like dark cherry | 1.3 | 3.0 | 3.7 | 4.7 | 4.7 | 2.0 |

TABLE 3

| | Sample No. | | | | |
|---|---|---|---|---|---|
| | 3-1 | 3-2 | 3-3 | 3-4 | 3-5 |
| Isoamyl acetate (mg/L) | 1.5 | 1.5 | 1.5 | 1.5 | 1.5 |
| a* value | 6 | 11 | 16 | 21 | 26 |
| Fragrance like dark cherry | 1.3 | 4.7 | 5.0 | 3.3 | 2.0 |
| Appearance of red beer | 2.3 | 4.0 | 5.0 | 5.0 | 5.0 |

### Study of Test Results: Examples

As is apparent from the results of Samples 2-2 to 2-5 and 3-2 to 3-4, it is confirmed that since the content of isoamyl acetate is 0.7 to 2.4 mg/L and the a* value is 7.0 to 21.0, the oxidized soy sauce odor is changed to the fragrance like dark cherry. On the other hand, from the results of Samples 2-1, 2-6, 3-1, 3-5, it is confirmed that the oxidized soy sauce odor is not changed to the fragrance like dark cherry, when the content of isoamyl acetate is too low or too high or when the a* value is too low or too high.

As a reference, Table 4 discloses L* value, a* value, b* value and content of isoamyl acetate of commercial products (beer-taste beverages having a red color) examined. The L* value, the a* value and the b* value were obtained by putting each commercial product in a 10 mm cell, to be measured by transparent object color measurement (according to instruction manual of CM-3600d) using the spectrophotometer (device name: CM-3600d manufactured by Konica Minolta, Inc.). The content of isoamyl acetate was measured using the gas chromatograph (device name: Agilent 6890 gas chromatograph) equipped with the FID detector, according to the method in “8.22 Low boiling aroma component” of BCOJ beer analysis method (published by the Brewing Society of Japan, edited by the Brewers Association International Technology Committee (Analysis Committee), 1 Apr. 1996).

TABLE 4

| Commercial | | | | |
|---|---|---|---|---|
| product No. | L* value | a* value | b* value | Isoamyl acetate (mg/L) |
| A | 66.0 | 21.4 | 82.5 | 1.2 |
| B | 80.1 | 5.5 | 54.0 | 0.4 |
| C | 61.6 | 26.0 | 83.9 | 0.4 |
| D | 61.4 | 26.3 | 83.9 | 0.4 |
| E | 54.8 | 24.2 | 71.4 | 1.9 |
| F | 79.6 | 6.6 | 58.2 | 1.8 |

As is apparent from the results in Table 4, there is no product satisfying the requirements of the present invention in the commercial products.

### REFERENCE SIGNS LIST

S1: pre-fermentation step
S2: fermentation step
S3: post-fermentation step

1. A beer-taste beverage, which has an a* value in an L*a*b* color system of 7.0 or more and 21.0 or less and a content of acetate ester of 0.7 mg/L or more and 2.4 mg/L or less.

2. The beer-taste beverage according to claim 1, wherein the a* value is 11.0 or more and 21.0 or less.

3. The beer-taste beverage according to claim 1, wherein the a* value is 7.0 or more and 16.0 or less.

4. The beer-taste beverage according to claim 1, wherein the content of the acetate ester is 1.0 mg/L or more and 2.0 mg/L or less.

5. The beer-taste beverage according to claim 1, wherein the acetate ester is isoamyl acetate.

6. The beer-taste beverage according to claim 1, wherein the beer-taste beverage has an L* value of 50.0 or more and 90.0 or less and a b* value of 50.0 or more and 90.0 or less.

7. The beer-taste beverage according to claim 1, wherein the beer-taste beverage contains no colorant.

8. The beer-taste beverage according to claim 1, wherein the beer-taste beverage has a malt ratio of 50 mass % or more.

9. A beer-taste beverage,
wherein the beer-taste beverage has an a* value in an L*a*b* color system of 11.0 or more and 16.0 or less, a content of isoamyl acetate of 1.5 mg/L or more and 2.0 mg/L or less, an L* value of 50.0 or more and 90.0 or less, a b* value of 50.0 or more and 90.0 or less,
the beer-taste beverage contains no colorant, and
the beer-taste beverage has a malt ratio of 50 mass % or more.

10. A method for producing the beer-taste beverage of claim 1, the method comprising:
producing the beer-taste beverage so that the beer-taste beverage has the a* value in an L*a*b* color system of 7.0 or more and 21.0 or less and the content of the acetate ester of 0.7 mg/L or more and 2.4 mg/L or less.

11. A method for improving aroma of a beer-taste beverage, the method comprising:
preparing the beer-taste beverage so that the beer-taste beverage has an a* value in an L*a*b* color system of 7.0 or more and 21.0 or less and content of acetate ester of 0.7 mg/L or more and 2.4 mg/L or less.
